(19) United States
(12) Patent Application Publication
(10) Pub. No.: US 2010/0184692 A1
(43) Pub. Date: Jul. 22, 2010

(54) Amidated Dopamine Neuron Stimulating Peptides for CNS Dopaminergic Upregulation

(75) Inventors: Don M. Gash, Lexington, KY (US); Greg A. Gerhardt, Nicholasville, KY (US); John D. Glass, Shoreham, NY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/447,213

(22) PCT Filed: Oct. 26, 2007
(86) PCT No.: PCT/US07/22696

§ 371 Date: Mar. 25, 2010

Related U.S. Application Data
(60) Provisional application No. 60/854,693, filed on Oct. 27, 2006.

(51) Int. Cl. A61K 38/10; C07K 7/08; C07K 7/06; A61K 38/08; A61P 25/00

(52) U.S. Cl. 514/13; 530/326; 530/330; 530/327; 514/15

(57) ABSTRACT

The present invention relates to novel proteins, referred to herein as amidated glial cell line-derived neurotrophic factor (GDNF) peptides (or “Amidated Dopamine Neuron Stimulating peptides (ADNS peptides)”), that are useful for treating brain diseases and injuries that result in dopaminergic deficiencies.

### FIELD OF THE INVENTION

The present invention relates to novel proteins, referred to herein as amidated glial cell line-derived neurotrophic factor (GDNF) peptides (or “Amidated Dopamine Neuron Stimulating peptides (ADNS peptides)”), that are useful for treating brain diseases and injuries that result in dopaminergic deficiencies.

### BACKGROUND OF THE INVENTION

Neurotrophic factors are endogenous proteins that modulate cell signaling pathways regulating stem cell proliferation, neuronal differentiation, differentiation, growth and regeneration (Barde Y., “Trophic factors and neuronal survival”, Neuron 2:1525-1534 (1989); Gotz, R., et al., “The conservation of neurotrophic factors during vertebrate evolution”, Comp Biochem Physiol Pharmacol Toxicol Endocrinol 108: 1-10 (1994); and Goldman, S. A., “Adult neurogenesis: from canaries to the clinic”, J Neurobiol 36: 267-86 (1998)). They are generally small, soluble proteins with molecular weights between 13 and 24 KDa and often function as homodimers. Because of this physiological role, neurotrophic factors are useful in treating the degeneration of nerve cells and the loss of differentiated function that occurs in a variety of neurodegenerative diseases.

Many neurotrophic factors are both neuroprotective (protecting neurons from injury) and neurorestorative (promoting structural and functional regeneration). The best defined protective functions are seen during neural development. During development, excessive numbers of neurons are generated in many brain regions. Developing neurons that fail to make connections with appropriate trophic factor producing target cells are deprived of necessary neurotrophic factors and die. Those neurons that establish connections survive and function properly (e.g. NGF; see Campenot, R. B. and MacInnis, B. L. “Retrograde transport of neurotrophins: fact and function”, J Neurobiol 58: 217-229 (2004)). Neurotrophic factors are also capable of promoting the re-growth of damaged neurons and their processes both in vitro and in animal models (see Lad, S. P. et al., “Individual and combined effects of TrkA and p75NTR nerve growth factor receptors: A role for the high affinity receptor site”, J Biol Chem 278: 24808-24817 (2003a) and Lad, S. P. et al., “Nerve growth factor: structure, function and therapeutic implications for Alzheimer's disease”, Curr Drug Targets CNS Neurol Disord 2: 315-334 (2003b)). Identifying neurotrophic factors with the right combination of protective and restorative actions and developing effective strategies for drug delivery have profound therapeutic implications for Parkinson's disease, Alzheimer's disease, Huntington's disease and other degenerative processes in the brain (including those induced by brain injury).

Glial cell line-derived neurotrophic factor (GDNF) is a trophic factor shown to dramatically protect and enhance the function of dopamine neurons in vitro and in vivo in rodents and monkeys (Beck, K. D., et al., “Mesencephalic dopaminergic neurons protected by GDNF from axotomy-induced degeneration in the adult brain”, Nature, 373:339-41 (1995); and Bjorklund, A., et al., “Towards a neuroprotective gene therapy for Parkinson's disease: use of adenovirus, AAV and lentivirus vectors for gene transfer of GDNF to the nigrostriatal system in the rat Parkinson model”, Brain Res., 886:82-98 (2000), Gash, D. M., et al., “Functional Recovery in Parkinsonian Monkeys Treated with GDNF, Nature”, 380:252-255 (1996); Grondin, R., et al., “Striatal GDNF Infusion Promotes Structural and Functional Recovery in Advanced Parkinsonian Monkeys. Brain”, 125:2191-2201 (2002); Grondin, R., et al., “GDNF Increases Stimulus-evoked Dopamine Release and Motor Speed in Aged Rhesus Monkeys”, J. Neurosci., 23:1974-1980 (2003); Hebert M. A., et al., “Functional Effects of GDNF in Normal Rat Striatum: Presynaptic Studies Using In Vivo Electrochemistry and Microdialysis”, J. Pharm. Exp. Ther., 279:1181-1190 (1996); Hebert M. A. and Gerhardt, G. A., “Behavioral and Neurochemical Effects of Intranigral GDNF Administration on Aged Fischer 344 Rats”, J. Pharm. Exp. Ther., 282:760-768 (1997); Hou, J. G. G., et al., “Glial Cell line-Derived Neurotrophic Factor Exerts Neurotrophic Effects on Dopaminergic Neurons In Vitro and Promotes Their Survival And Regrowth After Damage by 1-Methyl-4-Phenylpyridinium”, J. Neurochem., 66:74-82 (1996); Kordower, J. H., et al., “Clinicopathological Findings Following Intraventricular Glial-Derived Neurotrophic Factor Treatment in a Patient with Parkinson's Disease”, Ann Neurol., 46(3):419-424 (1999); Kordower, J. H., et al., “Neurodegeneration Prevented by Lentiviral Vector Delivery of GDNF in Primate Models of Parkinson's Disease”, Science, 290:767-773 (2000); Palfi, S., et al., “Lentivirally Delivered Glial Cell Line-derived Neurotrophic Factor Increases the Number of Striatal Dopaminergic Neurons in Primate Models of Nigrostriatal Degeneration”, J. Neurosci., 22:4942-4954 (2002); Tomac, A., et al., “Protection and Repair of the Nigrostriatal Dopaminergic System by GDNF In Vivo”, Nature, 373:335-339 (1995)).

The current standard treatment, levodopa, is palliative and does not prevent the relentless progression of Parkinson's degeneration. GDNF exerts effects on dopamine neurons that slow the process of Parkinson's disease and even reverses some of the degenerative changes. Preclinical studies conducted to date suggest that GDNF exerts at least three general trophic actions on dopamine neurons in the substantia nigra: pharmacological upregulation, restoration and neuroprotection. With regard to pharmacological upregulation, GDNF upregulates dopaminergic functions, such as increasing the evoked release of dopamine (Gerhardt, G. A. et al., “GDNF improves dopamine function in the substantia nigra but not the putamen of unilateral MPTP-lesioned rhesus monkeys”, Brain Res 817: 163-171 (1999) and Grondin et al., 2003). It also appears to modulate the phosphorylation of TH (Salvatore, M. et al. “Striatal GDNF administration increases tyrosine hydroxylase phosphorylation in the rat striatum and substantia nigra”. J Neurochem. 90:245-54., (2004)). With regard to restoration, GDNF increases the number of neurons expressing the dopamine markers TH and DAT in the substantia nigra (Gash et al., 1996; Kordower et al., 2000; and Grondin et al., 2002). This suggests that one trophic action is to stimulate recovery of injured/quiescent nigral neurons. Supporting this interpretation is the consistent observation that GDNF promotes increases in dopamine neuron perikaryal size and the number of neurites. With regard to neuroprotection, nigrostriatal administration of GDNF either shortly before or following a neurotoxic challenge (e.g. 6-OHDA, methyl-amphetamine or MPTP) protects dopamine neurons from injury in rodents and nonhuman primates (Kordower et al., 2000 and Fox, C. M., “Neuroprotective effects of GDNF against 6-OHDA in young and aged rats”, Brain Res 896:56-63 (2001)).

Accordingly, GDNF therapy is expected to be helpful in the treatment of nerve damage caused by conditions that compromise the survival and/or proper function of one or more types of nerve cells. Such nerve damage may occur from a wide variety of different causes. Nerve damage may occur to one or more types of nerve cells by, for example: (1) physical injury, which causes the degeneration of the axonal processes and/or nerve cell bodies near the site of injury; (2) temporary or permanent cessation of blood flow to parts of the nervous system, as in stroke; (3) intentional or accidental exposure to neurotoxins, such as chemotherapeutic agents (e.g., cisplatinum) for the treatment of cancer or dideoxycytidine (ddC) for the treatment of AIDS; (4) chronic metabolic diseases, such as diabetes or renal dysfunction; or (5) neurodegenerative diseases such as Parkinson's disease, Alzheimer's disease, and Amyotrophic Lateral Sclerosis (ALS), which result from the degeneration of specific neuronal populations.

GDNF therapy may be particularly helpful in the treatment of neurodegenerative conditions involving the degeneration of the dopaminergic neurons of the substantia nigra, such as Parkinson's disease. The expected impact of GDNF therapy is not just to produce an increase in the dopaminergic neurotransmission at the dopaminergic nerve terminals in the striatum (which will result in a relief of the symptoms), but also to slow down, or even stop, the progression of the degenerative processes and to repair the damaged nigrostriatal pathway and restore its function. GDNF may also be used in treating other forms of damage to or improper function of dopaminergic nerve cells in human subjects. Such damage or malfunction may occur in schizophrenia and other forms of psychosis. The only current treatments for such conditions are symptomatic and require drugs which act upon dopamine receptors or dopamine uptake sites, consistent with the view that the improper functioning of the dopaminergic neurons which innervate these receptor-bearing neuronal populations may be involved in the disease process.

However, initial clinical trials involving ventricular delivery of GDNF showed no statistically significant differentiation of the placebo and active treatment groups (Nutt, J. G. et al. “Randomized, double-blind trial of glial cell line-derived neurotrophic factor (GDNF) in PD”, Neurology 60: 69-73 (2003)), perhaps because insufficient amounts of GDNF reached critical target sites from the CSF (Ai, Y. et al., “Intraputamenal Infusion in Aging Rhesus Monkeys: Distribution and Dopaminergic Effects”, J Comp Neurol 461: 250-26125 (2003); and Kordower, J. H., et al. (2000)). In addition, a phase 2 trial evaluating intraputamenal delivery of glial cell line-derived neurotrophic factor (GDNF) for the treatment of Parkinson's disease failed to achieve its primary end point, a 25% improvement on the Unified Parkinson Disease Rating Scale (UPDRS) motor score “off” medication after six months of treatment (Lang, A. E. et al., “Randomized controlled trial of intraputamenal glial cell line-derived neurotrophic factor infusion in Parkinson's disease”, Ann Neurol 59:459-466 (2006)). There are strong indications from studies in rhesus monkeys using the same delivery system and protocol followed in the phase 2 study that drug bioavailability significantly contributed to the failure of the trial (Salvatore et al., “Point source concentration of GDNF may explain failure of phase II clinical trial”. Exp Neurol 2006, in press). The concentration of GDNF around the catheter tip and limited diffusion into surrounding brain parenchyma was limited to a brain volume representing 2-9% of the human putamen. Thus GDNF distribution in the phase 2 trial was likely limited to a small brain region, and could affect only a limited segment of the brain undergoing parkinsonian degeneration.

Successful trophic factor therapy requires site-specific delivery and distribution of the trophic factor throughout the target tissue (the putamen for Parkinson's disease). The blood brain barrier effectively blocks entry from blood borne proteins, including trophic factors. Infusions into the cerebrospinal fluid are not effective in humans because of brain size and may produce unwanted side effects by stimulating other trophic factor responsive populations such as sensory neurons.

In addition to focal delivery into the appropriate site, the delivery must be tightly regulated. Regardless of the method used to deliver GDNF (i.e., direct infusion, stem cells, encapsulated cells, gene therapy) prolonged elevated levels of GDNF in the brain outside of the target area may produce adverse side-effects. Circulating antibodies to GDNF are one possible outcome and it is quite typical to find antibodies to endogenous proteins used therapeutically (e.g. beta interferon and insulin, see Durelli, L., et al., “Anti-interferon antibodies in multiple sclerosis: molecular basis and their impact on clinical efficacy”, Front Biosci 9: 2192-2204 (2004) and Stoever, J. A. et al., “Inhaled insulin and insulin antibodies: a new twist to an old debate”, Diabetes Technol Ther 4: 157-161 (2002)). The effects of circulating GDNF antibodies are not known. Focal Purkinje cell lesions have been reported in some monkeys receiving high levels of GDNF in a toxicology study (see Sherer, T. B., et al., Movement Dis 21:136-141 (2006)). Another possible side-effect is aberrant sprouting and tyrosine hydroxylase downregulation of the nigrostriatal dopaminergic pathway in rats exposed to high GDNF levels from viral vector gene transfer (Georgievska, B., et al. “Neuroprotection in the rat Parkinson model by intrastriatal GDNF gene transfer using a lentiviral vector”, Neuroreport 13: 75-82 (2002)). Also, increased neuronal death has been reported in rats with elevated GDNF from viral vector gene transfer in a stroke model (Arvidsson, A. et al., “Elevated GDNF levels following viral vector-mediated gene transfer can increase neuronal death after stroke in rats”, Neurobiol Dis 14: 542-556, (2003)).

While GDNF has not met the criteria for clinical efficacy in the two phase 2 trials conducted to date (Nutt et al., 2003; Lang et al., 2006), it appears to be the most potent dopaminergic trophic factor found to date. Thus, the ideal drug for treating Parkinson's disease and other neurodegenerative processes in the brain would possess the positive trophic actions of GDNF. Delivery could be targeted to the appropriate brain area by any of a number of methods including direct infusion, viral vectors or even nasal sprays. In particular, biologically active peptides with trophic actions may offer many of the desired properties. To date, such biologically active peptides have not been identified.

A crude peptide extract from the brain cerebrolysin has been tested in human studies, with modest effects reported (Lukhanina, E. P. et al., “Effect of cerebrolysin on the electroencephalographic indices of brain activity in Parkinson's disease”, Zh Nevrol Psikhiatr Im S S Korsakova 104: 54-60 (2004)). Three small molecule compounds have also been tested in Parkinson's disease patients: the tripeptide glutathione, tocopherol, and Coenzyme Q10 (Weber, C. A., et al. “Antioxidants, supplements and Parkinson's disease”, Ann Pharmacother 40: 935-938 (2006)). The three small molecule compounds also appear to have only minor benefits for patients.

Consequently, there continues to exist a long-felt need for effective agents and methods for the treatment and prevention of brain diseases and injuries that result in dopaminergic deficiencies. Accordingly, it is an object of the present invention to provide agents for treating and preventing such diseases and injuries in a subject, comprising novel amidated GDNF-derived peptides that have dopaminergic trophic factor activity. This and other such objectives will be readily apparent to the skilled artisan from this disclosure.

### SUMMARY OF THE INVENTION

In one embodiment, the present invention provides a composition comprising at least one of the following peptides: (a) a purified Amidated Dopamine Neuron Stimulating peptide (ADNS peptide) comprising the amino acid sequence ERNRQAAAANPENSRGK-amide; (b) a purified ADNS peptide comprising the amino acid sequence FPLPA-amide; and (c) a purified ADNS peptide comprising the amino acid sequence PPEAPAEDRSL-amide. In another embodiment, the present invention provides a method for treating a brain disease or injury resulting in dopaminergic deficiencies, comprising administering a pharmaceutically effective amount of the composition to a subject in need thereof, wherein said composition further comprises at least one of a pharmaceutically acceptable vehicle, excipient, and diluent. Preferably, the subject is a mammal, and most preferably, the subject is human.

### BRIEF DESCRIPTION OF THE FIGURES

FIG. 1 depicts the amino acid sequence of mature human GDNF.

FIG. 2 depicts the post-translational processing of splice form 1 of human GDNF.

FIG. 3 depicts the precursor segments of the ADNS peptides ERNRQAAAANPENSRGK-amide; FPLPA-amide; and PPEAPAEDRSL-amide.

FIG. 4 depicts the average K⁺-evoked release of dopamine in Fischer 344 rats treated with ADNS peptides.

FIG. 5 depicts the increase in major metabolites of dopamine in Fischer 344 rats treated with ADNS peptides.

FIG. 6 depicts the increased survival of dopamine neurons in cell cultures treated with ADNS peptides.

FIGS. 7A and B depict the results of in vivo microdialysis used to investigate the dynamics of dopamine release in the basal ganglia of the right putamen following treatment with ADNS peptides.

FIGS. 8A-F depict the histopathological response to the injection of ADNS peptides in the nigral region using standard histochemical techniques.

FIGS. 9A-D depict a series of photomicrographs evaluating the substantia nigra compacta (SNc) containing the population of dopamine neurons that degenerates in Parkinson's disease after treatment with ADNS peptides.

### DETAILED DESCRIPTION OF THE INVENTION

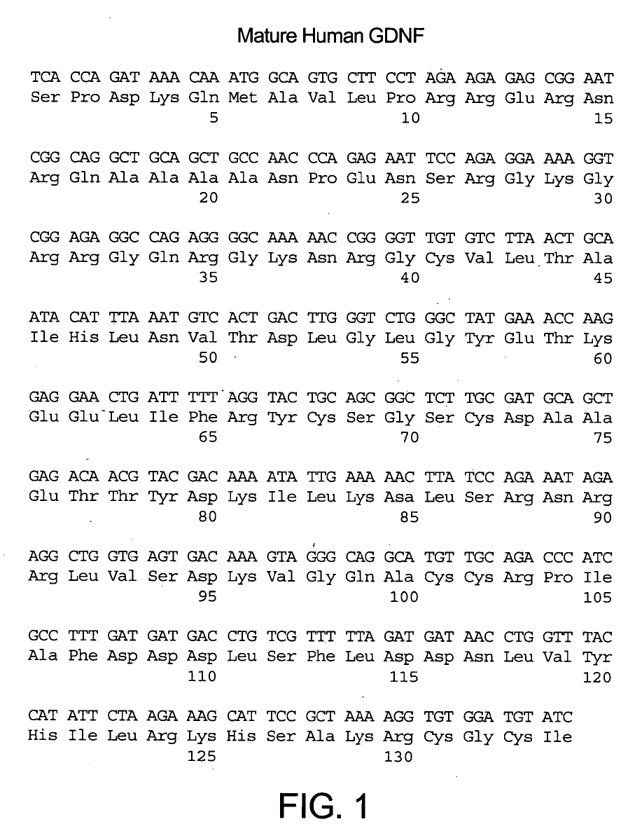

Human glial cell line-derived neurotrophic factor (hGDNF) is synthesized as a precursor that is processed and secreted as a mature protein of 134 amino acids. Mature human GDNF has the amino acid sequence depicted in FIG. 1 (SEQ ID NO:1).

The present invention is related to the realization that human GDNF (splice form 1) precursor protein conforms to a rather exacting sequence profile characteristic of neuropeptide precursor proteins. GDNF is expressed in at least three isoforms that result from alternative splicing of mRNA. Proteins expressed from these RNA splice variants differ in their N-terminal regions (see NCBI entry NP 000505 for isoform 1; NCBI entry NP 964701 for isoform 2; and NCBI entry NP 954704 for isoform 3). Isoforms 1 and 2 are secretory protein precursors with N-terminal signal peptides. Isoform 3 is likely a nuclear-targeted protein with a nuclear localization signal (NLS), but no signal peptide. The three isoforms are differentially expressed, apparently under regulatory control.

All of the isoforms contain the sequence that is considered mature GDNF with “full biological activity” (residues 78-211 in isoform 1). In fact, recombinant proteins further truncated at the N-terminus are purported to have the “full biological activity”. From the fact that three separate precursors of the same GDNF molecule are expressed under separate regulation, two with signal peptides and the third with a probable nuclear localization signal, suggests that there are both nuclear and cell surface receptors for GDNF.

The separately regulated expression of two different secretory isoforms (isoforms 1 and 2) suggests a biologically significant function for the different N-terminal sequences of the isoforms. The conventional wisdom is that residues 20-77 of isoform 1 precursor constitute a “domain propeptide”. The present invention provides a new interpretation of the importance of the 20-77 “domain propeptide” region of isoform 1 precursor. The “domain propeptide” segment of isoform 1 precursor is the metabolic precursor of two small amidated peptides, according to well established enzymatic pathways for release of peptide amide hormones and neuropeptides from their precursor proteins. The two small amidated peptides are FPLPA-amide and PPEAPAEDRSL-amide.

Furthermore, a third small amidated peptide, ERNRQAAAANPENSRGK-amide, may be released from consensus enzymatic processing of residues 88-110 of the isoform 1 precursor. This consensus peptide amide precursor occupies the N-terminal sequence of “mature GDNF”, which is presumably not critical or at least includes residues that are not critical for biological activity, according to the patent literature. The isoform 2 precursor contains the sequence FPLPA, but does not contain the amidation signal (GKR, residues 25-27) in the case of Isoform 1 precursor. Isoform 2 is not, in other words, a potential precursor of FPLPAamide according to known enzymatic pathways. Isoform 3 does not contain the sequence FPLPA at all, so likewise can not be a metabolic precursor of FPLPA-amide. Both Isoforms 1 and 2 are consensus precursors of ERNRQAAAANPENSRGK-amide, but isoform 3 is not.

Thus, human GDNF isoform 1 is a secretory protein that can potentially yield three small amidated peptides by consensus enzymatic pathways known to release peptide amide hormones and neuropeptides from their known precursor proteins. This is a rare combination of enzymatic processing motifs and strongly suggests that GDNF isoform 1 precursor is the metabolic precursor of up to three small ADNS peptides, in addition to the larger C-terminal domain that is widely supposed to possess the “full biological activity” of GDNF. C-terminal amidation in natural peptides is highly correlated with receptor mediated signal transduction: about half of the known peptide hormones and neuropeptides and C-terminal amidation is rare and almost unknown among other peptides of biological origin.

Animal studies with synthetic peptides corresponding to the consensus products of Isoform 1 precursor protein are consistent with some or all of these peptides being biologically active and involved in regulation of dopamine metabolism. This is to be expected from a protein that yields multiple biologically active regulatory peptides in a fixed molar ratio. The fact that the different Isoforms of GDNF precursor protein are consensus precursors of different combinations of amidated peptides in addition to GDNF, suggests a reason for differential expression of three separate isoforms (in addition to differential secretory and nuclear routing).

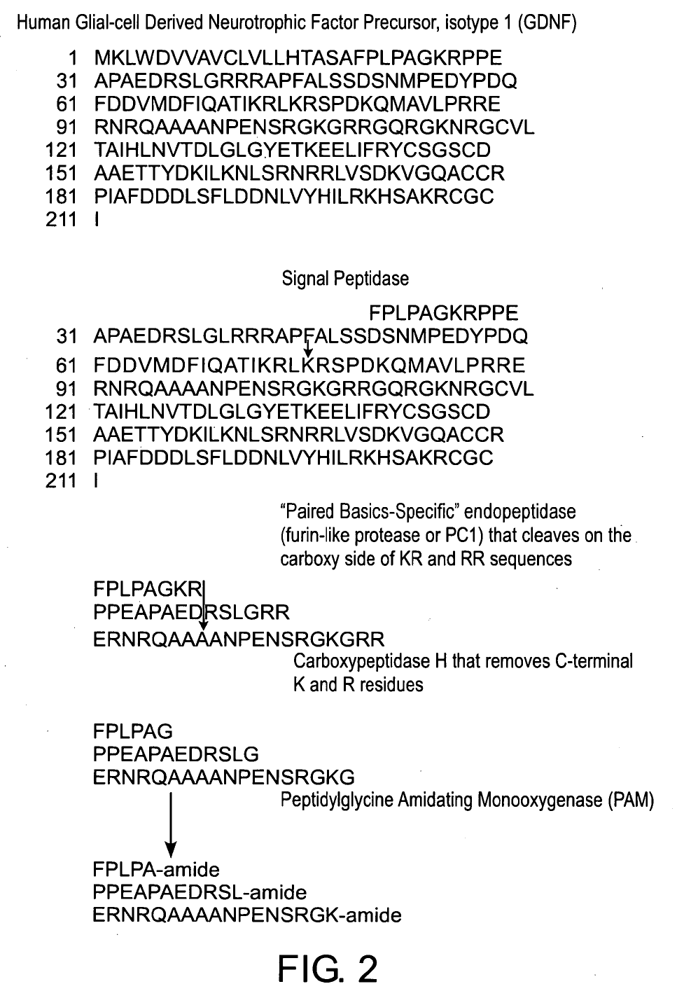

According to this model, spice form 1 of human GDNF may be post-translationally processed in vivo to yield three small amidated peptides, as indicated in FIG. 2. These small amidated peptides may mediate some or all of the biological effects of GDNF. The present invention is based on the unexpected discovery that these small amidated fragments of the mature GDNF protein retain the biological activity of GDNF.

Thus, the ADNS peptides of the present invention include these three small amidated peptides, which are represented by the amino acid sequences ERNRQAAAANPENSRGK-amide; FPLPA-amide; and PPEAPAEDRSL-amide (SEQ ID NOs:2, 3, and 4, respectively).

The ADNS peptides of the present invention are promising candidates for treatment and prevention of neurodegenerative conditions involving dopaminergic deficiencies, such as Parkinsons disease, age-associated motor and cognitive slowing, and other diseases and injuries to the brain. The small ADNS peptides are easily within the range of synthetic production methods, so that the molecules could be subjected to rigorous structure-activity studies to optimize pharmacological activities and biostability. In addition, as the difficulties in delivering GDNF clinically may well be related to the fact that recombinant GDNF is not properly processed into active forms, the small ADNS peptides may overcome some of these difficulties. Finally, small peptides are generally much less antigenic than proteins and can be synthesized free of the trace host protein contaminants always present in recombinant proteins.

The small ADNS peptides of the present invention include biologically active synthetic or recombinant ADNS peptides, ADNS peptides produced from GDNF, biologically active ADNS peptide variants (including insertion, substitution, and deletion variants), and chemically modified derivatives thereof. Also included are biologically active ADNS peptide variants that are substantially homologous to any one of the ADNS peptides having the amino acid sequence set forth in SEQ ID NOs:2, 3, or 4.

The term “biologically active” as used herein means that the ADNS peptide demonstrates similar neurotrophic properties, but not necessarily all of the same properties, and not necessarily to the same degree, as the GDNF protein having the amino acid sequence set forth in SEQ ID NO:1. The selection of the particular neurotrophic properties of interest depends upon the use for which the ADNS peptide is being administered. The ADNS peptides are biologically active and demonstrate dopaminergic neuron survival characteristics similar to that demonstrated by the combination of amide peptides represented by SEQ ID NOs:2, 3, or 4 using the evaluation of dopamine neuron survival in cultures of newborn rat midbrain dopamine neurons as an exemplary bioassay, as discussed in the examples below.

The term “substantially homologous”, as used herein, means a degree of sequence homology to any one of the ADNS peptides having the amino acid sequences set forth in SEQ ID NOs:2, 3, or 4 that is preferably at least 70%, most preferably at least 80%, and even more preferably at least 90% or even 95%.

As used herein, the term “ADNS peptide,” “peptide amide,” or “amidated peptide” means a peptide comprising the group —CONH₂ at the C-terminal end. This amidation occurs in vivo, once the peptides are formed by the enzyme, peptidylglycine amidating monooxygenase (PAM). The ADNS peptides of the present invention can be readily obtained in a variety of ways, including, without limitation, recombinant expression, purification from natural sources, and/or chemical synthesis. Preferably, the ADNS peptides can be chemically synthesized by commercial venders. The ADNS peptides used in the present examples were synthesized using tBOC chemistry and at a single scale range (which generates a theoretical crude yield of 500-1,000 mg for a 10-20 mer respectively), by the Keck Biotechnology Resource Laboratory at Yale University (New Haven, Conn.).

ADNS peptide pharmaceutical compositions typically include a therapeutically effective amount of at least one of a ADNS peptide represented by SEQ ID NOs:2, 3, and 4 in admixture with one or more pharmaceutically and physiologically acceptable formulation materials. Suitable formulation materials include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. For example, a suitable vehicle may be water for injection, physiological saline solution, or artificial cerebrospinal fluid (CSF), possibly supplemented with other materials common in compositions for parenteral administration. Neutral buffered saline or saline mixed with serum albumin are further exemplary vehicles.

The primary solvent in a vehicle may be either aqueous or non-aqueous in nature. In addition, the vehicle may contain other pharmaceutically-acceptable excipients for modifying or maintaining the pH, osmolarity, viscosity, clarity, color, sterility, stability, rate of dissolution, or odor of the formulation. Similarly, the vehicle may contain still other pharmaceutically-acceptable excipients for modifying or maintaining the stability, rate of dissolution, or rate of release of ADNS peptide, or for promoting the absorption or penetration of ADNS peptide across the blood-brain barrier. Such excipients are those substances usually and customarily employed to formulate dosages for parenteral administration in either unit dose or multi-dose form or for direct infusion into the CSF by continuous or periodic infusion from an implanted pump.

Once the therapeutic composition has been formulated, it may be stored in sterile vials as a solution, suspension, gel, emulsion, solid, or dehydrated or lyophilized powder. Such formulations may be stored either in a ready to use form or in a form, e.g., lyophilized, requiring reconstitution prior to administration.

The optimal pharmaceutical formulation will be determined by one skilled in the art depending upon the route of administration and desired dosage. See for example, Remington's Pharmaceutical Sciences, 18th Ed. (1990, Mack Publishing Co., Easton, Pa. 18042) pages 1435-1712 the disclosure of which is hereby incorporated by reference. The composition may also involve particulate preparations of polymeric compounds such as polylactic acid, polyglycolic acid, etc. or into liposomes. Hylauronic acid may also be used, and this may have the effect of promoting sustained duration in the circulation. Such compositions may influence the physical state, stability, rate of in vivo release, and rate of in vivo clearance of the present proteins and derivatives.

Other effective administration forms, such as parenteral slow-release formulations, inhalant mists, orally active formulations, or suppositories, are also envisioned. Preferred ADNS peptide pharmaceutical compositions are formulated for parenteral administration, e.g., intracerebroventricular injection. Such parenterally administered therapeutic compositions are typically in the form of a pyrogen-free, parenterally acceptable aqueous solution comprising ADNS peptide in a pharmaceutically acceptable vehicle. One preferred vehicle is physiological saline.

It is also contemplated that certain formulations containing ADNS peptides are to be administered orally. ADNS peptide which is administered in this fashion may be encapsulated and may be formulated with or without those carriers customarily used in the compounding of solid dosage forms. The capsule may designed to release the active portion of the formulation at the point in the gastrointestinal tract when bioavailability is maximized and pre-systemic degradation is minimized. Additional excipients may be included to facilitate absorption of ADNS peptide.

Diluents, flavorings, low melting point waxes, vegetable oils, lubricants, suspending agents, tablet disintegrating agents, and binders may also be employed.

The ADNS peptides may be administered parenterally via a subcutaneous, intramuscular, intravenous, transpulmonary, transdermal, intrathecal or intracerebral route. ADNS peptides that do not cross the blood-brain barrier may be given directly intracerebrally or otherwise in association with other elements that will transport them across the barrier. It is preferred that the ADNS peptide is administered intracerebroventricularly or into the brain or spinal cord subarachnoid space. ADNS peptides may also be administered intracerebrally directly into the brain parenchyma. Slow-releasing implants in the brain containing the neurotrophic factor embedded in a biodegradable polymer matrix can also deliver ADNS peptides. ADNS peptides may be administered extracerebrally in a form that has been modified chemically or packaged so that it passes the blood-brain barrier, or it may be administered in connection with one or more agents capable of promoting penetration ADNS peptide across the barrier. For example, a conjugate of NGF and monoclonal anti-transferrin receptor antibodies has been shown to be transported to the brain via binding to transferrin receptors. To achieve the desired dose of ADNS peptide, repeated daily or less frequent injections may be administered, or truncated ADNS peptide may be infused continuously or periodically from a constant- or programmable-flow implanted pump. The frequency of dosing will depend on the pharmacokinetic parameters of ADNS peptide as formulated, and the route of administration.

Regardless of the manner of administration, the specific dose is typically calculated according to body weight or body surface area. For diseases involving the brain, the specific dose is typically calculated according to the approximate brain weight of the subject, which also may be estimated based on body weight or body surface area. Further refinement of the calculations necessary to determine the appropriate dosage for treatment involving each of the above mentioned formulations is routinely made by those of ordinary skill in the art, especially in light of the dosage information and assays disclosed herein. Appropriate dosages may be ascertained through use of the established assays for determining dosages utilized in conjunction with appropriate dose-response data. The final dosage regimen involved in a method of treating a specific condition will be determined by the attending physician, considering various factors which modify the action of drugs, e.g., the age, condition, body weight, sex and diet of the subject, the severity of any infection, time of administration and other clinical factors.

ADNS peptides of the present invention may also be employed, alone or in combination with other growth factors in the treatment of nerve disease. For example, ADNS peptides may be used in treating some forms of nerve disease in combination with nerve growth factor. In addition, other factors or other molecules, including chemical compositions, may be employed together with ADNS peptides. In the treatment of Parkinson's Disease, it is contemplated that ADNS peptide be used by itself or in conjunction with the administration of Levodopa, wherein the ADNS peptide would enhance the production of endogenous dopamine and the neuronal uptake of the increased concentration of dopamine.

As stated above, it is also contemplated that additional neurotrophic or neuron nurturing factors will be useful or necessary to treat some neuronal cell populations or some types of injury or disease. Other factors that may be used in conjunction with ADNS peptides include, but are not limited to: mitogens such as insulin, insulin-like growth factors, epidermal growth factor, vasoactive growth factor, pituitary adenylate cyclase activating polypeptide, interferon and somatostatin; neurotrophic factors such as brain derived neurotrophic factor, neurotrophin-3, neurotrophin-4/5, neurotrophin-6, insulin-like growth factor, ciliary neurotrophic factor, acidic and basic fibroblast growth factors, fibroblast growth factor-5, transforming growth factor-.beta., cocaine-amphetamine regulated transcript (CART) and mature GDNF; and other growth factors such as epidermal growth factor, leukemia inhibitory factor, interleukins, interferon, and colony stimulating factors; as well as molecules and materials which are the functional equivalents to these factors.

It is envisioned that the continuous administration or sustained delivery of a ADNS peptide may be advantageous for a given treatment. While continuous administration may be accomplished via a mechanical means, such as with an infusion pump, it is contemplated that other modes of continuous or near continuous administration may be practiced. For example, chemical derivatization may result in sustained release forms of the protein which have the effect of continuous presence in the blood stream, in predictable amounts, based on a determined dosage regimen. Thus, ADNS peptides of the present invention include ADNS peptides derivatized to effectuate such continuous administration.

ADNS peptide cell therapy, e.g., intracerebral implantation of cells producing ADNS peptides, is also contemplated. This embodiment of the present invention may include implanting into subject's cells which are capable of synthesizing and secreting a biologically active form of the ADNS peptides of the present invention. Such ADNS peptide producing-cells may be cells which do not normally produce a neurotrophic factor but have been modified to produce ADNS peptides, or they could be cells whose ability to produce GDNF has been augmented by transformation with a polynucleotide suitable for the expression and secretion of ADNS peptides. In order to minimize a potential immunological reaction in subjects, it is preferred that the cells be of human origin.

Implanted cells may be encapsulated to avoid infiltration of the cells into brain tissue. Human or non-human animal cells may be implanted in subjects in biocompatible, semi-permeable polymeric enclosures or membranes to allow release of a ADNS peptide, but that prevent destruction of the cells by the subject's immune system or by other detrimental factors from the surrounding tissue. Alternatively, the subject's own cells, transformed ex vivo to produce ADNS peptides, could be implanted directly into the subject without such encapsulation.

The methodology for the membrane encapsulation of living cells is familiar to those of ordinary skill in the art, and the preparation of the encapsulated cells and their implantation in subjects may be accomplished. See for example, U.S. Pat. Nos. 4,892,538; 5,011,472; and 5,106,627, the disclosures of which are hereby incorporated by reference. A system for encapsulating living cells is also described in PCT Application WO 91/10425 of Aebischer et al., specifically incorporated herein by reference. See also, PCT Application WO 91/10470 of Aebischer et al.; Winn et al., Exper. Neurol., 113:322-329, 1991; Aebischer et al., Exper. Neurol., 111:269-275, 1991; and 35 Tresco et al., ASAIO, 38:17-23, 1992, the disclosures of which are hereby incorporated by reference.

ADNS peptide gene therapy in vivo is also envisioned, wherein a nucleic acid sequence encoding a ADNS peptide is introduced directly into the subject. For example, a nucleic acid sequence encoding a ADNS peptide is introduced into target cells via local injection of a nucleic acid construct with or without an appropriate delivery vector, such as an adeno-associated virus vector. Alternative viral vectors include, but are not limited to, retrovirus, adenovirus, herpes simplex virus and papilloma virus vectors. Physical transfer may be achieved in vivo by local injection of the desired nucleic acid construct or other appropriate delivery vector containing the desired nucleic acid sequence, liposome-mediated transfer, direct injection (naked DNA), receptor-mediated transfer (ligand-DNA complex), or microparticle bombardment (gene gun). It should be noted that the ADNS peptide formulations described herein may be used for veterinary as well as human applications and that the term “subject” should not be construed in a limiting manner. In the case of veterinary applications, the dosage ranges should be the same as specified above.

As a means of further characterizing the ADNS peptides of the present invention, antibodies can be developed which bind to the ADNS peptides. One of ordinary skill in the art can use well-known, published procedures to obtain monoclonal and polyclonal antibodies, or recombinant antibodies, which specifically recognize and bind to the various ADNS peptides of the present invention.

Other aspects and advantages of the present invention will be understood upon consideration of the following illustrative examples. The examples are not to be construed in any way as limiting the scope of this invention.

### Examples

### Example 1

### BLAST Analysis

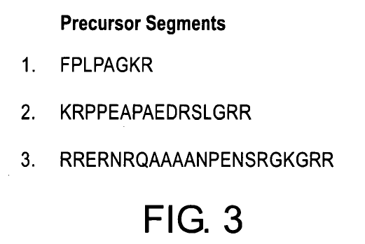

The three hypothetical precursor segments (RRERNRQAAAANPENSRGKGRR; FPLPAGKR; and KRPPEAPAEDRSLGRR; see FIG. 3) were subjected to BLAST searches for short, nearly identical sequences. RRERNRQAAAANPENSRGKGRR is present in GDNF splice forms 1 and 2. There are some sequence variations by species, but consensus post-translational processing signals are maintained across species. FPLPAGKR was found to be invariant in the available GDNF splice form 1 sequences, but does not occur in splice forms 2 and 3. KRPPEAPAEDRSLGRR scored hits in splice forms 1, but not in the other GDNF splice forms. There are some sequence variations by species, but consensus processing signals are maintained across species. Thus, these ADNS peptides are unique to mostly spice form 1 of GDNF and not other splice forms of the pre pro GDNF.

### Example 2

### Synthesis of ADNS Peptides

Three peptides, designated GER9263, GER9264, and GDR9265 (see Table 1, below) were synthesized by Keck Biotechnology Resource Laboratory, Yale University New Haven, Conn. Synthetic peptides can be made routinely up to 40 residues and often, depending on sequence, up to 70 residues by this facility. All peptides were chromatographed on a preparative C-18 or C-4 RP-HPLC system and delivered as a lyophilized material. Yields for normal peptides under 40 residues were “guaranteed” at 50 mg or more and at 90+% purity. Yields and purity are often higher, varying with the peptide sequence and length. All peptides made in the Keck facility at the 0.5 mmole scale are done by tBOC chemistry and at a single scale range (which generates a theoretical crude yield of 500-1,000 mg for a 10-20 mer respectively). The purified peptides were characterized by analytical RP-HPLC, amino acid analysis, and FAB mass spectroscopy.

TABLE 1
ADNS peptides

| Peptide | Sequence |
|---|---|
| GER9263 | ERNRQAAAANPENSRGK-amide |
| GER9264 | FPLPA-amide |
| GDR9265 | PPEAPAEDRSL-amide |

### Example 3

### Dopaminergic Activity of ADNS Peptides in Fischer 344 Rats

Experiments were performed to test the effects of the three ADNS peptides (GER9263, GER9264, and GDR9265) on dopaminergic activity in normal young adult male Fischer 344 rats. The peptides were combined in a 1:1:1 ratio. There were three test groups with six animals per group: vehicle (citrate buffer), vehicle plus 30 μg peptide mixture, and vehicle plus 100 μg peptide mixture. The vehicle or vehicle plus peptide solutions were steriotaxically injected in equal portions into two sites each in the right substantia nigra.

One month after drug administration, the basal levels of dopamine and dopamine metabolites were measured by microdialysis in the right straitum. Potassium and amphetamine evoked release of dopamine were also evaluated.

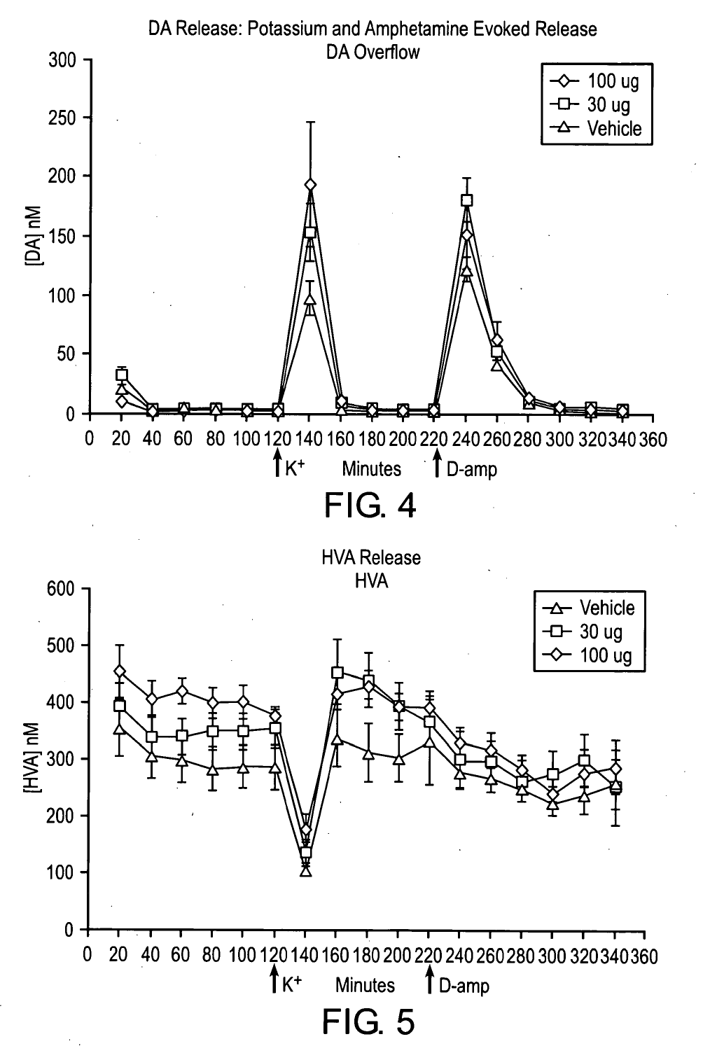

While basal levels of dopamine were not significantly altered in the striatum, average K⁺-evoked release of dopamine increased by over 50% (FIG. 4). Average amphetamine-evoked release was more variable, but ranged from about 30% for 100 μg to about 45% for 30 μg. While basal dopamine levels were about the same in all three groups, basal metabolite levels were elevated. As shown in FIG. 5, the major metabolites 3,4-Dihydroxy-Phenylacetic Acid (DOPAC) and Homovanillic Acid (HVA) were increased by about 40% (DOPAC) and 20-40% (HVA), depending upon the dose.

### Example 4

### Dopaminergic Activity of ADNS Peptides in Cell Cultures of Rat Midbrain Dopamine Neurons

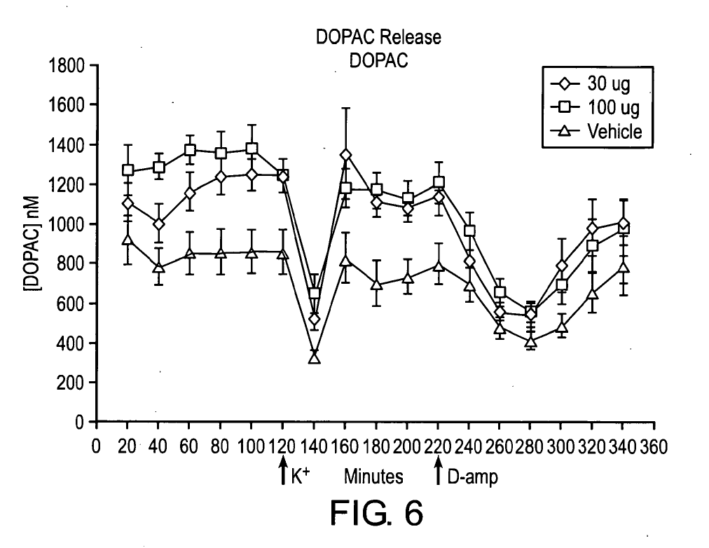

To further characterize the bioactivity of the peptides, blinded samples were sent to Dr. Michael Zigmond at the University of Pittsburgh for testing in cultures of newborn rat midbrain dopamine neurons. Peptide mixture samples (GER9263, GER9264, and GDR9265 combined in a 1:1:1 ratio) were compared to citrate buffer alone, two different lots of GDNF, and Neurturin (FIG. 6). Both lots of GDNF and the 100 ng/ml ADNS peptide mixture exerted about the same effects in dopamine neuron survival, increasing survival by about 25-30% over vehicle levels. The peptide mixture at a concentration of 50 ng/ml promoted an increase in survival of around 10%, approximately the same effect as 100 ng/ml Neurturin, a trophic factor that is closely related to GDNF.

### Example 5

### ADNS Peptides and Response to CNS Delivery in an Aged Rhesus Monkey

A study was performed demonstrating that a mixture of three ADNS peptides (GER9263, GER9264, and GDR9265 combined in a 1:1:1 ratio) exerts pharmacological effects on CNS nigral dopamine neurons in an aged rhesus monkey similar to those produced by GDNF. Marked increases of 68-125% in stimulus-evoked dopamine release were measured in the putamen. Motor speed, as measured in fine motor hand movements, increased by up to 58%, into the range of young adult monkeys. General body movements increase by 38%, indicating much higher activity levels. The effects from unilateral treatment were long-lasting (for at least one month) and bilateral, similar to those resulting from GDNF treatment. Histopathological analysis of the injection sites in the substantia nigra revealed only mild, circumscribed pathology from the peptide injections. The pharmacological effects of ADNS peptides on upregulating nigrostriatal dopamine system functions are extraordinary and suggest their potential therapeutic use for the treatment of Parkinson's disease and age-associated movement dysfunctions.

The ADNS peptides tested in this Example are three amidated peptides predicted to exert potent biological effects similar to those of Glial Cell Line-Derived Neurotrophic Factor (GDNF). The effects of GDNF on CNS dopamine neurons fall generally into three categories: pharmacological upregulation of dopaminergic activity, neuronal regeneration and neuroprotection. This study was designed to assess the pharmacological effects of ADNS peptides on substantia nigra dopamine neurons in the nonhuman primate brain. It was previously shown that CNS delivery of GDNF increases stimulus-evoked dopamine release in aged rhesus monkeys (Grondin et al., 2003). In addition, behavioral correlates of increased dopaminergic activity were recorded in these animals, improved motor functions and increased motor speed. The present study was a case report on one aged monkey that received a 100 μg injection of ADNS peptides into the substantia nigra of the brain and was followed for 30 days. Motor speed was measured weekly using an automated movement analysis panel (MAP) and EthoVision, a video tracking program. Movement features were rated weekly using a nonhuman primate clinical rating scale (Zhang, Z. et al., “Motor slowing and parkinsonian signs in aging rhesus monkeys mirror human aging.”, J Gerontology: Biol. Sci. 55A:B473-B480, (2000)). Stimulus-evoked dopamine release was analyzed by microdialysis at the 30 day time point. The animal was then euthanized and the brain recovered for histopathological analysis.

A thirty-four year old female rhesus monkey (ID# NJ05) weighing 7 kg was used. The animal was diagnosed as having an inoperable mammary tumor, with the attending veterinarian suggesting that the monkey be placed in a terminal study and euthanized for humanitarian reasons. The animal was maintained on a 12-hour light/12-hour dark cycle and housed individually in a cage measuring 9 square feet, with an elevated perch, front access doors and side rear access doors connecting the housing cage to an adjacent activity module. The diet consisted of certified primate biscuits given in the morning (7:30 AM), and supplemented daily in the afternoon (1:30 PM) with fresh fruit or vegetables. Water was available ad libitum.

All procedures were conducted in the Laboratory Animal Facilities of the University of Kentucky, which are fully accredited by the Association for Assessment and Accreditation of Laboratory Animal Care. Veterinarians skilled in the health care and maintenance of nonhuman primates supervised all animal care.

Magnetic resonance images (MRI) were obtained on a I.5T clinical imager (Siemens Magnetom Vision) using a standard cross polarized extremity coil. After being sedated with ketamine hydrochloride (˜20 mg/kg; i.m.) plus atropine sulfate (˜0.04 mg/kg; i.m.), the animal was anesthetized with sodium pentobarbital (˜10 mg/kg; i.v.) and imaged to provide stereotaxic coordinates for peptide delivery. The animal's head was positioned in the extremity coil using an MRI compatible stereotaxic head frame. This frame kept the animal's head level and immobilized at the center of the radio frequency coil using ear and mouth bars. An initial set of coordinates was taken using the earbars, toothbar and a zeroing bar targeted to the gum line between the two upper middle incisors to allow replication any time the animal was replaced in the stereotaxic apparatus.

The coil/frame assembly was then positioned to place the animal's head at magnet isocenter. Sets of T1-weighted 3D-FLASH images were collected for determination of the brain coordinates (TR/TE=22/9 ms; FA=35°; FOV=96×96×90 mm; Matrix=128×128×90; Nacq=2; TA=6 min 48 sec). All images were acquired as coronal slices relative to the brain. Antero-posterior, lateral and vertical coordinates for stereotaxic surgery were derived from the T1-weighted coronal brain images. The interaural line was identified on the scans by modified earbars containing Vitamin E. This provided a precise reference-point, which allowed for anteroposterior measurements to the target. Lateral measurements were determined by measuring the distance from the sagittal sinus/third ventricle to the target site. Vertical measurements were determined from the surface of the brain to the target at the lateral coordinate.

Following sedation with ketamine hydrochloride (˜20 mg/kg; i.m.) plus atropine sulfate (˜0.04 mg/kg; i.m.), the animal was intubated via the orotracheal method and intravenous lines secured. Then, the animal was anesthetized with isoflurane (1-3%) and placed in an MRI compatible Kopf stereotaxic apparatus in a ventral-lateral position as per the coordinates previously (see Anatomical MRI above). The animal was maintained on a heated blanket and had cardiac and respiratory parameters monitored during the procedure, which was carried out using sterile field conditions. Coordinates for peptides injections were determined by MRI prior to the surgery as described above. After being shaved, the scalp area was cleaned using antiseptic procedures with sterile 4×4 sponges soaked in Betadine® surgical scrub followed by 70% isopropyl alcohol. This procedure was repeated. After the alcohol dries, Betadine® prep was applied and the animal was covered with sterile drapes. Then, an incision was made through the scalp and the skin and muscles overlying the skull were reflected. Small holes were drilled in the skull directly over the target area. The overlying meninges were removed to expose the surface of the brain.

A 1.0 mg/ml concentration of the three-peptide mixture in citrate buffer was used. It was sterilized by filtration prior to injection Using MRI-guided procedures, a 27 G needle coupled to a Hamilton syringe containing 100 μl (i.e. 100 μg) of the peptide mixture was lowered in the right rostral SNc (AP: 11, L: 5, DV: 30 from surface of the brain). A volume of 50 μl was delivered using a nanopump at a rate of 1 μl/min. This was followed by a 20 min waiting period before retracting the needle out of the brain (1 mm/min). The needle/syringe assembly was then moved 1 mm caudally and 0.5 mm laterally and lowered into the more caudal SNc (AP: 10, L: 5.5, DV: 29 from surface of the brain). This was followed by a 20 min waiting period before retracting the needle out of the brain (1 mm/min). After completion of the injections, the scalp incision was sutured over the exposed areas per normal procedures and the animal was given an analgesic (buprenorphine, 0.01 mg/kg, i.m.). Vital signs were monitored until the animal awakened, at which point the animal was covered with warm blankets and taken back to its cage and monitored until it was ambulatory.

To assess changes in motor functions, the animal was videotaped prior to injecting the peptide mixture, and weekly after the injection for four weeks. The videotaping cage measured 28 inW×32 inH×32 inD, had a white background wall and a clear Lexan window permitting videotaping. The videotaping cage was illuminated by two 48 in-long fluorescent lights located 30 in above. Water (via a cage attached bottle) was available ad libitum through out the session. The animal was fed fruit or vegetables after completion of data acquisition and upon return to its home-cage.

Beginning at 1 PM, a technician entered the room and placed small food items (e.g. gummy bears) on the ledge of the cage to elicit the animal to stand-up and reach out for the food. Then, the animal was videotaped for 30 minutes with no one in the room. Following this 30-minute videotaping period, the technician re-entered the room, stopped recording and attached a non-wired version of the monkey Movement Analysis Panel (MAP, see below) to the doorway of the cage for a hand retrieval test. Five to six preferred small food items (e.g. miniature marshmallows) were placed on each side of the panel. The animals was given 10 minutes (default time) to retrieve the food items, at which point the tester re-entered the room and stopped recording. This portion of the session was videotaped, with a focus on the hands. Starting at 2 PM, the same procedures described above were repeated. At the end of the testing session, the animal was returned to its home cage.

As described above, standardized videotaping procedures were conducted pre- and post-treatment. Behavioral parameters associated with motor function were scored from coded videotapes from 0 (normal) to 3 (severe disability) in the following categories: rigidity, bradykinesia, posture, balance, tremor, and hand dexterity (see Zhang et al., 2000). Rigidity is defined as a decrease in limb extension and/or use. Motor dysfunctions were rated in half-point increments by an experienced rater.

Distance traveled (cm) was quantified from 8-mm videotapes (SONY P6-120MPL) using a commercially available video tracking system EthoVision Pro (version 2.3, Noldus Information Technology, Asheville, N.C.) coupled to a SONY Digital 8 video cassette recorder. This system runs on a Pentium based computer with a frame grabber card (PICOLO, Belgium), so that the analog video signal coming from the video cassette recorder is digitized and transferred to the computer. A window on the computer screen directly displays the video image, and the boundaries within which tracking took place were defined by accurately tracing the outline of the cage in the video image, in addition, two zones were outlined, so that the overall activity measured in the entire cage could be analyzed in terms of vertical (top half) or horizontal (bottom half) activity. As described above, the animal was videotaped for 60-minute periods, pre- and post-treatment, and the video tracks were analyzed at a rate of 6-sample/sec. For every sample, the cage was scanned and the position of the tracked animal was determined by using a gray scale detection method (brightness). This entails calibrating the software to distinguish the dark-colored animal from the background, which is then defined as all other pixels. The back wall of the cage was painted white to provide a background with a maximum degree of contrast with the dark-colored primate. This automated method relies on determining the position of the center of mass of the animal in the cage, and the resultant x-y coordinates extracted as a function of time are used for calculating the movement pattern during the observation period. These coordinates were subsequently related to actual spatial measures by calibrating the software to the dimension (width) of the cage, the distance traveled by the animal were calculated in centimeters instead of pixels.

Movement Analysis Panel (MAP). In addition, fine/hand motor movement times in retrieving food items from a platform level placed in a receptacle chamber were measured using an automated clear Lexan MAP attached to the door opening of the home cage (see Gash, D. M. et al. “An automated movement analysis panel for upper limb motor functions in rhesus monkeys and humans”, J. Neurosci Methods 89:111-117, (1999) and Zhang et al., 2000). The receptacle chamber is divided into left and right halves, and is accessible on each side through two portals (armhole portal and receptacle portal). Movement times were measured by arrays of three photodiodes around each portal that automatically relayed to the computer when one or more beams were broken by passage of the monkey's arm/hand. Testing was conducted in the afternoon, prior to injecting the peptide mixture, and weekly thereafter for four weeks. Fresh fruit and vegetables were given to the animal after completion of the testing session. A day's testing session consisted of twelve trials, six on each side alternating between the right and left hand.

The animal underwent bilateral putamenal dialysis one month post ADNS peptide injection. Using the method described below, in vivo microdialysis procedures had also been conducted previously in the right striatum of the same animal (date: Feb. 10, 2004; coordinates: AP: 20.5 mm, L: 10.2 mm, DV from cortex: 21 mm).

Following normal MRI-guided stereotactic procedures (see Surgical procedures for ADNS peptide injection), custom-made CMA/11 dialysis probes with a membrane length of 3 mm and diameter of 0.24 mm (CMA Microdialysis, North Chelmsford, Mass.) were positioned (1 mm/min) bilaterally in the putamen (AP: 20 mm, L: 10.5 mm, DV from cortex: 20 mm). Probes were perfused continuously at a rate of 1.2 μl/min with artificial cerebrospinal fluid using a computerized multisyringe pump (World Precision Instruments). Microdialysate fractions were collected at 30 min intervals.

Following a 1-hour application of artificial cerebrospinal fluid to collect baseline fractions, excess potassium (100 mM KCl, 47.7 mM NaCl) was then included in the perfusate for a single 30-min fraction (t0-t30). Two hours later 250 μM amphetamine was included in the perfusate for a single 30-min fraction (t1-t150). Three additional fractions were collected after discontinuing amphetamine administration (t180-t240). The incision was then closed as per normal surgical procedures. Microdialysate fractions were analyzed using standard high performance liquid chromatography procedures coupled with electrochemical detection.

The tissue biopsies were collected for possible future use. At the end of the dialysis session, the animal was immediately euthanized. The deeply anesthetized animal (2 ml pentobarbital, i.v.) was transcardially perfused with heparinized ice-cold saline (6 L). Then, the brain was removed quickly, and dropped into a container of cold saline, which was placed on wet ice and taken back to the laboratory. Using an ice-cold mold, the brain was sectioned into 4-mm coronal slabs, rostral of the midbrain.

Multiple tissue punches were taken bilaterally from frozen 4-mm thick coronal tissue section using a 14 G biopsy needle in the caudate nucleus (n=18 per side, tissue slabs #3, #4 and #5), putamen (n=18 per side, tissue slabs #4, #5, #6), nucleus accumbens (n=7 per side, tissue slabs #3 and #4) and globus pallidus (n=5 per side, tissue slabs #6). Separate needles were used for the right and left (green tape) hemisphere. All punches were rapidly transferred to pre-weighed storage tubes, weighed once more, and stored at −80° C. Pictures of the punched slabs were taken to document the punching pattern, and then stored at −80° C.

The midbrain was taken out as a block, which included the cerebellum, and post-fixed for quantitative immunocytochemistry of substantia nigra dopamine neurons and TH+ processes. To do so, the midbrain was immediately immersed in 4% paraformaldehyde in a 0.1 M phosphate buffer (pH 7,4) at 4° C. for three days and transferred to 15% sucrose solution until saturated for sectioning. Then, a series of 40 μm-thick coronal sections were cut on a frozen sliding microtome. One out of every 12th adjacent sections was processed for cresyl violet (Nissl) and hemotoxylin and eosin (H & E) staIning. Also, 1/12th adjacent sections were processed for the following staining: a) 1:1000 mouse anti-glia fibrillary acidic protein (GFAP) antibody for reactive astrocytes, b) 1:200 mouse anti-HLA-DR antibody for reactive microglia, and c) 1:1000 mouse anti-tyrosine hydroxylase (TH) antibody for dopaminergic neurons (see Ai et al., 2003).

The animal recovered without problems from peptides delivery into the right substantia nigra. No clinically observable abnormal behaviors (e.g. auto-mutilation, stereotypic movements, dyskinesia, vomiting) were noted throughout the 30-day study.

As described above, standardized videotaping procedures were conducted pre- and post-treatment to assess changes in motor functions from coded videotapes. Prior to injecting the three-peptide mixture into the right substantia nigra, the animal was given a cumulative score of 3.25 points on the rating scale (See Table 2). Although the effect was variable, this score improved (the lower the score, the better the movement functions) by over 30% (i.e. 1 point) by the fourth week of the study. Similarly, distance traveled (cm) measured over weekly 60-minute periods using the automated video-tracking system (EthoVision) improved up to 38% by the fourth week post treatment versus baseline locomotor activity (from 7064 cm to 9780 cm). Last but not least, MAP performance times for left hand motor were 56% faster by week four post peptides treatment (from 0.81 sec to 0.36 sec). Right hand performance times were already faster than the left pre-peptide treatment. They further improved in the 4 weeks post-treatment, with performance times 14% faster (from 0.36 sec to 0.31 sec).

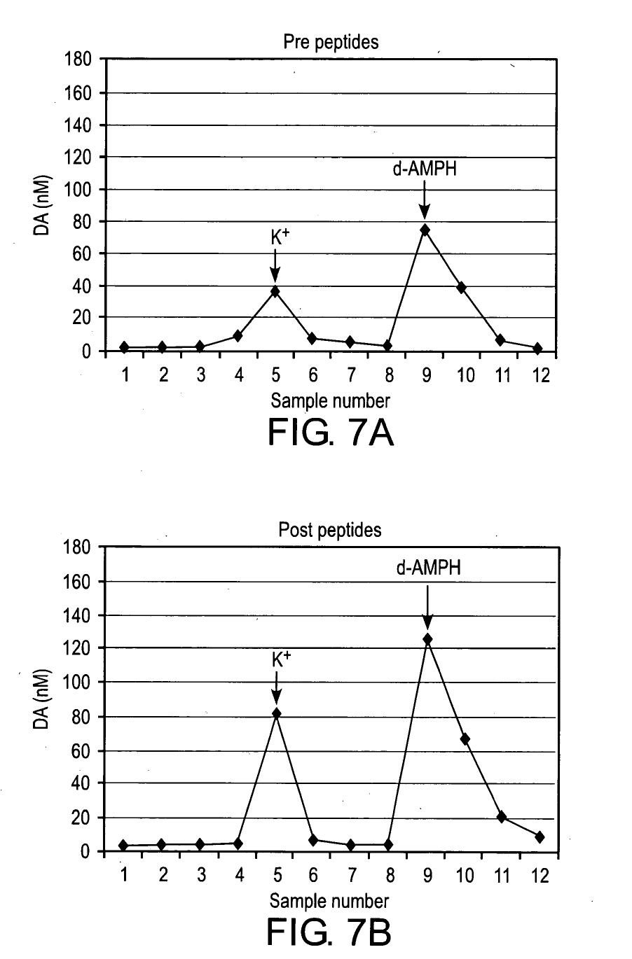

In vivo microdialysis was used to investigate the dynamics of dopamine release in the basal ganglia. Measurements were carried out in the right putamen four weeks post peptide treatment (see FIGS. 7A and B). In particular, FIG. 7 shows the results of potassium (K+) and d-amphetamine (d-AMPH) evoked release of dopamine measured in the right putamen of NJ05 using microdialysis to collect samples for neurochemical analysis. FIG. 7A shows the first recordings made Feb. 10, 2004, several years prior to the ADNS peptide study. In aging monkeys, dopamine evoked release to K+ and d-AMPH normally decreases with increasing age. FIG. 7B shows the second set of recordings made thirty days after ADNS peptide injections into the right substantia nigra on Apr. 27, 2006. K+ evoked release was increased from the 2004 reading from 36.5 nM to 82 nM. D-AMPH evoked release of dopamine increased from 74 nM to 125 nM after ADNS peptide administration. Thus, in comparison to measurements recorded under similar conditions two years earlier in the same animal, potassium-evoked overflow of dopamine was increased by 125% at 30 days post ADNS peptide administration (from 36.5 nM to 82 nM). Similarly, amphetamine-induced overflow of dopamine was increased by 68% compared to measurements recorded two years earlier (from 74 nM to 125 nM).

Basal levels of dopamine and dopamine metabolites in the striatum were determined from measurements in the dialysates prior to potassium and amphetamine stimulation. Basal dialysate levels of dopamine and HVA showed small changes from the baseline levels two years earlier (Table 2). However, basal levels of extracellular DOPAC were increased by 230% at thirty days post peptides injection compared to the earlier baseline measures (Table 3).

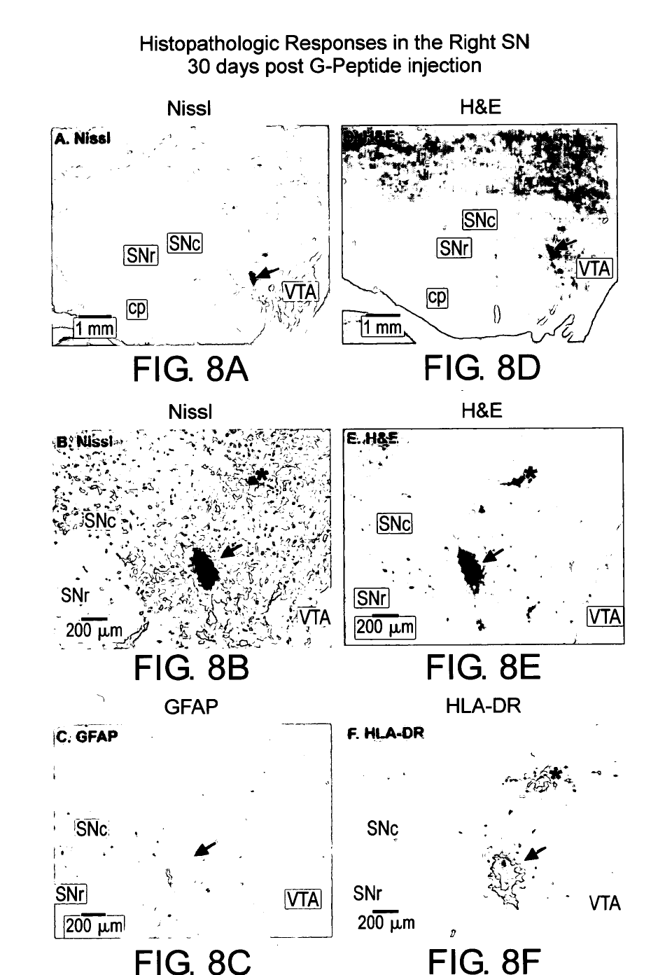

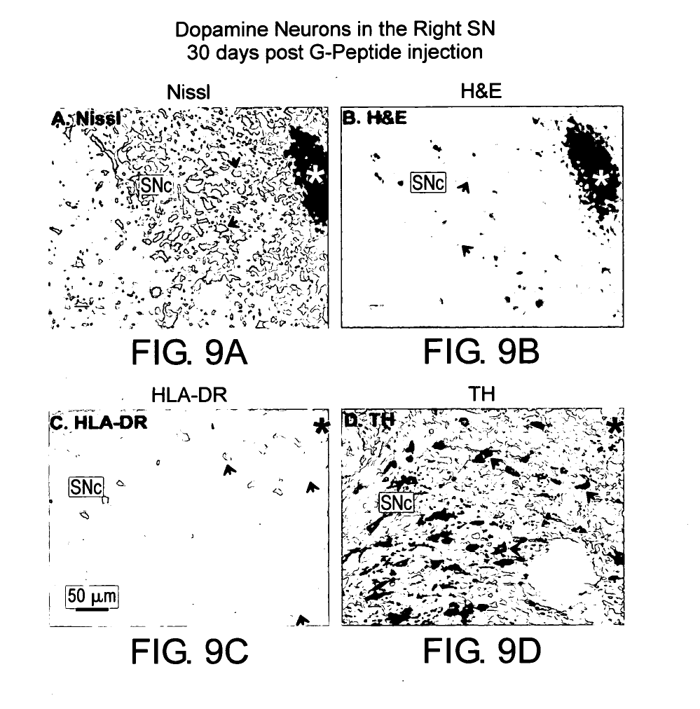

The histopathological response to the injection of peptides in the nigral region was mild (see FIGS. 8 and 9). In FIG. 8, one of the injection sites (arrow) is shown using standard histochemical techniques for Nissl staining and hematoxylin and eosin (H & E) staining. Immunostaining was conducted to assess the response of astrocytes (GFAP positive cells) and microglia (HLA-DR positive cells) to the injections. In FIGS. 8A and B, this injection site (arrow) was just dorsal to the substantia nigra, compacta (SNc). While there is an evident response at the injection site and a smaller satellite area (*) of reactivity dorsal to the main area, the injury response is very circumscribed. FIG. 8C shows that reactive astrocytosis, as assessed by GFAP positive reactivity around the needle track, is minimal, approximately that expected from a needle tract injury alone. In FIGS. 8D and E, the injury response appears similar using H & E to that seen with Nissl. The injury response is localized and does not seem to involve adjacent cells. Reactive microglia (HLA-DR+cells) are a prominent constituent of the injury response (arrow and *, FIG. 8F). (In FIG. 8, Cerebral peduncle=CP; Substantia nigra reticulatia=SNr; Ventral Tegmental Area=VTA. Scale bars are included in each photomicrograph.) The injection site showed reactive cells in an area about 200 μm wide by 400 μm long in Nissl-stained and H & E-stained sections (FIGS. 8A, B, D and E). The response consisted of activated microglia (HLA-DR positive cells, FIG. 8F). The absence of pronounced GFAP immunostaining (FIG. 8) indicated that the injection did not stimulate a glial reaction.

The substantia nigra compacta (SNc) containing the population of dopamine neurons that degenerates in Parkinson's disease is evaluated in the series of photomicrographs set forth in FIG. 9. In FIG. 9A, Nissl-rich neurons (arrowheads) adjacent to the peptide injection site (*) appear normal with prominent nuclei evident in the nucleus of some cells. Nissl staining corresponds to the presence of rough endoplasmic reticulum in the cytoplasm and indicates cells actively synthesizing protein. The H & E stained section in FIG. 9B also shows neurons with normal features (arrowheads) adjacent to the injection site (*). FIG. 9C shows that there are only a few scattered activated microglia (arrowheads showing HLA-DR positive cells) in the SNc. Large numbers would indicate ongoing pathological processes. Tyrosine hydroxylase (TH) is the rate-limiting enzyme in dopamine synthesis. The TH positive cells shown in FIG. 9D are dopamine neurons. Their size and exuberant expression of TH positive processes are indicative of healthy, active cells. Scale bars are included in each photomicrograph. Dopamine neurons in the substantia nigra appeared to be normal (FIG. 9). Tyrosine hydroxylase immunostaining (FIG. 9D) revealed large dopamine neuron perikarya (cell bodies) with extensive neuritic processes. Along with the Nissl and H & E stained sections (FIGS. 9A and B), the nigral cells showed features characteristic of healthy neurons. A few activated microglial cells (HLA-DR positive cells, FIG. 9C) were present in the nigral region, a typical feature of this brain region in healthy aged monkeys. Large numbers of activated microglia would be indicative of an ongoing disease process.

The subject in this study was a very old rhesus monkey, 34 years old. One year for a rhesus monkey is roughly equivalent to three years of human life, making this animal equivalent to a 100 year old person. The monkey was used in this study because it had a terminal disease, mammary cancer. The closest comparable monkeys in an earlier study treated with GDNF were 22-24 years old (Grondin et al., 2003). They received infusions of GDNF into the brain for 24 weeks while NJO5 had a single injection of an ADNS peptide mixture and was monitored for one month. Despite the differences, many of the responses were comparable to those seen to GDNF. NJO5 motor performance times on the MAP improved within four weeks on both the right (14%) and left (56%) sides. The improved motor speeds approached the speeds of aged monkeys receiving GDNF and those of normal young adult animals. Consistent with increased motor speed, general locomotor activity was increased by 38% by four weeks of treatment in NJO5. There were neurochemical changes in NJO5's brain along with the behavioral improvements. Both potassium and amphetamine evoked release were increased (125% and 68%, respectively) in the putamen in comparison to pretreatment levels. This was similar to the increased evoked release of dopamine in response to the same stimulants in aged rhesus monkeys treated with GDNF (Grondin et al., 2003). Basal dialysate levels of dopamine, HVA and DOPAC showed high variability, but were not significantly changed in aged GDNF recipients. NJO5's basal dopamine and dopamine metabolite levels were in the same range as the GDNF and vehicle-treated old animals. The only dramatic change seen in this animal was in DOPAC levels, which increased over three-fold post ADNS peptide treatment. The significance of this response is that it reflects higher levels of dopamine metabolism in the striatum.

Histopathology was much less extensive than the damage in the same region from the infusion of GDNF (see Gash, D. M. et al., “Trophic factor distribution predicts functional recovery in parkinsonian monkeys”, Ann Neurol 58(2):224-33 (2005)). The dopamine neurons in the substantia nigra appeared healthy, with numerous neuritic processes.

TABLE 2
Changes in motor functions post ADNS peptides delivery into the
right substantia nigra

| | | Locomotor | | Right MAP |
|---|---|---|---|---|
| | Disability | Activity | Left Map | times |
| | Score (pts)/% | (cm)/% | Times (sec)/% | (sec)/% |
| Treatment | baseline | baseline | baseline | baseline |
| Baseline | 3.25 | 7064 | 0.81 | 0.36 |
| Week 1 | 2.00/−38% | 8422/+19% | 0.86/+6% | 0.45/+25% |
| Week 2 | 2.00/−38% | 7136/+1% | 1.09/+35% | 0.39/+8% |
| Week 3 | 2.50/−23% | 6368/−10% | 0.75/−7% | 0.34/−6% |
| Week 4 | 2.25/−31% | 9780/+38% | 0.36/−56% | 0.31/−14% |

TABLE 3
Changes in basal dialysate levels of dopamine and dopamine
metabolites in the right putamen, 30 days post ADNS peptides
delivery into the right substantia nigra

| | Dopamine | HVA | DOPAC |
|---|---|---|---|
| Hemisphere | (nM) | (nM) | (nM) |
| Pre-peptides | 5.6 | 5033 | 127 |
| Post-peptides | 4.9 | 4191 | 419 |

While the present invention has been described with reference to specific embodiments, this application is intended to cover those various changes and substitutions that may be made by those of ordinary skill in the art without departing from the spirit and scope of the appended claims.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed. All publications cited herein are hereby incorporated by reference in their entirety.

1. A composition comprising at least one of the following peptides:
(a) a purified Amidated Dopamine Neuron Stimulating peptide (ADNS peptide) comprising the amino acid sequence ERNRQAAAANPENSRGK-amide;
(b) a purified ADNS peptide comprising the amino acid sequence FPLPA-amide; and
(c) a purified ADNS peptide comprising the amino acid sequence PPEAPAEDRSL-amide.

2. The composition of claim 1, comprising:
(a) a purified ADNS peptide comprising the amino acid sequence ERNRQAAAANPENSRGK-amide.

3. The composition of claim 1, comprising:
(b) a purified ADNS peptide comprising the amino acid sequence FPLPA-amide.

4. The composition of claim 1, comprising:
(c) a purified ADNS peptide comprising the amino acid sequence PPEAPAEDRSL-amide.

5. The composition of claim 1, comprising:
(a) a purified ADNS peptide comprising the amino acid sequence ERNRQAAAANPENSRGK-amide; and
(b) a purified ADNS peptide comprising the amino acid sequence FPLPA-amide.

6. The composition of claim 1, comprising:
(a) a purified ADNS peptide comprising the amino acid sequence ERNRQAAAANPENSRGK-amide; and
(c) a purified ADNS peptide comprising the amino acid sequence PPEAPAEDRSL-amide.

7. The composition of claim 1, comprising:
(b) a purified ADNS peptide comprising the amino acid sequence FPLPA-amide; and
(c) a purified ADNS peptide comprising the amino acid sequence PPEAPAEDRSL-amide.

8. The composition of claim 1, comprising:
(a) a purified ADNS peptide comprising the amino acid sequence ERNRQAAAANPENSRGK-amide;
(b) a purified ADNS peptide comprising the amino acid sequence FPLPA-amide; and
(c) a purified ADNS peptide comprising the amino acid sequence PPEAPAEDRSL-amide.

9. A method for promoting the survival and/or function of dopaminergic neurons, comprising administering a composition according to claim 1.

10. A method for treating a brain disease or injury resulting in dopaminergic deficiencies, comprising administering a pharmaceutically effective amount of a composition according to claim 1 to a subject in need thereof, wherein said composition further comprises at least one of a pharmaceutically acceptable vehicle, excipient, and diluent.

11. A composition comprising at least one of the following peptides:
(a) a purified ADNS peptide comprising an amino acid sequence that is substantially homologous to the amino acid sequence ERNRQAAAANPENSRGK-amide;
(b) a purified ADNS peptide comprising an amino acid sequence that is substantially homologous to the amino acid sequence FPLPA-amide; and
(c) a purified ADNS peptide comprising an amino acid sequence that is substantially homologous to the amino acid sequence PPEAPAEDRSL-amide, wherein:
said purified ADNS peptides (a), (b), and (c) are biologically active.


SEQUENCE LISTING (15 sequences)

SEQ ID NO: 1 - protein, 134 aa, Homo sapiens
SPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 2 - protein, 17 aa, Artificial Sequence
  Description of Artificial Sequence Synthetic peptide
ERNRQAAAANPENSRGK

SEQ ID NO: 3 - protein, 5 aa, Artificial Sequence
  Description of Artificial Sequence Synthetic peptide
FPLPA

SEQ ID NO: 4 - protein, 11 aa, Artificial Sequence
  Description of Artificial Sequence Synthetic peptide
PPEAPAEDRSL

SEQ ID NO: 5 - DNA, 402 bp, Homo sapiens
  CDS (1)..(402)
tcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatc

SEQ ID NO: 6 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 7 - protein, 192 aa, Homo sapiens
FPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 8 - protein, 8 aa, Artificial Sequence
  Description of Artificial Sequence Synthetic peptide
FPLPAGKR

SEQ ID NO: 9 - protein, 14 aa, Artificial Sequence
  Description of Artificial Sequence Synthetic peptide
PPEAPAEDRSLGRR

SEQ ID NO: 10 - protein, 20 aa, Artificial Sequence
  Description of Artificial Sequence Synthetic peptide
ERNRQAAAANPENSRGKGRR

SEQ ID NO: 11 - protein, 6 aa, Artificial Sequence
  Description of Artificial Sequence Synthetic peptide
FPLPAG

SEQ ID NO: 12 - protein, 12 aa, Artificial Sequence
  Description of Artificial Sequence Synthetic peptide
PPEAPAEDRSLG

SEQ ID NO: 13 - protein, 18 aa, Artificial Sequence
  Description of Artificial Sequence Synthetic peptide
ERNRQAAAANPENSRGKG

SEQ ID NO: 14 - protein, 16 aa, Artificial Sequence
  Description of Artificial Sequence Synthetic peptide
KRPPEAPAEDRSLGRR

SEQ ID NO: 15 - protein, 22 aa, Artificial Sequence
  Description of Artificial Sequence Synthetic peptide
RRERNRQAAAANPENSRGKGRR